(19) United States
(12) Patent Application Publication
(10) Pub. No.: US 2025/0230498 A1
(43) Pub. Date: Jul. 17, 2025

(54) SPATIALLY IDENTIFYING NUCLEIC ACIDS THAT INTERACT WITH PROTEINS

(71) Applicant: 10x Genomics, Inc., Pleasanton, CA (US)

(72) Inventor: Layla Katiraee, Castro Valley, CA (US)

(21) Appl. No.: 19/015,911

(22) Filed: Jan. 10, 2025

Related U.S. Application Data
(60) Provisional application No. 63/619,929, filed on Jan. 11, 2024.

(51) Int. Cl. C12Q 1/6874; C12Q 1/37; C12Q 1/6804

(52) U.S. Cl. CPC C12Q 1/6874; C12Q 1/37; C12Q 1/6804; G01N 2333/96411; G01N 2333/96416

(57) ABSTRACT

Provided herein are methods, compositions, and kits to spatially detect a polypeptide-nucleic acid complex or a protein-nucleic acid complex in a biological sample. For example, such methods can include contacting binding agents with a biological sample, wherein a binding agent specifically binds a polypeptide of a polypeptide-nucleic acid complex; aligning the biological sample with a substrate comprising a plurality of capture moieties, wherein the binding agent interacts with a capture moiety; releasing the nucleic acid of the binding agent-polypeptide-nucleic acid complex; hybridizing the released nucleic acid to a capture domain of a capture probe on an array; and using determined sequences of a spatial barcode in the capture probe and the released nucleic acid to determine the location of the released nucleic acid in the biological sample, thereby determining the location of the polypeptide-nucleic acid complex in the biological sample.

### CROSS-REFERENCE TO RELATED APPLICATIONS

This application claims the benefit of priority to U.S. Provisional Patent Application No. 63/619,929, filed on Jan. 11, 2024, the contents of which are hereby incorporated by reference.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Generally, spatial analysis takes advantage of targeting a particular analyte such as mRNA in a sample using a capture probe with a poly(T) capture domain. However, this approach is incapable of isolating polypeptide-nucleic acid complexes. One option to spatially identify polypeptide-nucleic acid complexes is by using multiple substrates. For example, one substrate (e.g., a slide) can comprise capture moieties while another substrate includes an array with barcoded capture probes. This set up provides the ability to capture binding agent-polypeptide-nucleic acid complexes on one substrate (e.g., the substrate including capture moieties) and releasing and capturing the nucleic acid on the spatially barcoded array. There is a need for improved spatial analysis methods in which the spatial location of a polypeptide-nucleic acid complex, including protein-nucleic acid complexes, can be determined in a tissue or other biological sample.

### SUMMARY

This disclosure features methods of isolating one or more polypeptides that interact with a nucleic acid to determine the location of the polypeptide-nucleic acid complex in a biological sample. Provided herein are methods of determining a location of a polypeptide-nucleic acid complex in a biological sample, the method comprising: (a) contacting a plurality of binding agents with the biological sample, wherein a binding agent of the plurality of binding agents specifically binds a polypeptide of the polypeptide-nucleic acid complex thereby forming a binding agent-polypeptide-nucleic acid complex and wherein the binding agent comprises a binding moiety; (b) aligning the biological sample with a first substrate comprising a plurality of capture moieties such that at least a portion of the biological sample is aligned with at least a portion of the substrate comprising the plurality of capture moieties, wherein the binding moiety of the binding agent interacts with a capture moiety of the plurality of capture moieties; (c) providing an array comprising a plurality of capture probes, wherein a capture probe of the plurality of capture probes comprises: (i) a spatial barcode, and (ii) a capture domain; (d) releasing the nucleic acid of the binding agent-polypeptide-nucleic acid complex, thereby generating a released nucleic acid; (e) hybridizing the released nucleic acid to the capture domain of the capture probe; and (f) determining (i) the sequence of the spatial barcode, or a complement thereof, and (ii) all or a portion of the sequence of the released nucleic acid, or a complement thereof, and using the determined sequences of (i) and (ii) to determine the location of the released nucleic acid in the biological sample, thereby determining the location of the polypeptide-nucleic acid complex in the biological sample.

Also provided herein are methods of determining a location of a protein-nucleic acid complex in a biological sample, the method comprising: (a) contacting a plurality of binding agents with the biological sample, wherein a binding agent of the plurality of binding agents specifically binds a protein of a protein-nucleic acid complex thereby forming a binding agent-protein-nucleic acid complex and wherein the binding agent comprises a binding moiety; (b) aligning the biological sample with a first substrate comprising a plurality of capture moieties such that at least a portion of the biological sample is aligned with at least a portion of the substrate comprising the plurality of capture moieties, wherein the binding moiety of the binding agent interacts with a capture moiety of the plurality of capture moieties; (c) providing an array comprising a plurality of capture probes, wherein a capture probe of the plurality of capture probes comprises: (i) a spatial barcode, and (ii) a capture domain; (d) releasing the nucleic acid of the binding agent-protein-nucleic acid complex, thereby generating a released nucleic acid; (e) hybridizing the released nucleic acid to the capture domain of the capture probe; and (f) determining (i) the sequence of the spatial barcode, or a complement thereof, and (ii) all or a portion of the sequence of the released nucleic acid, or a complement thereof, and using the determined sequences of (i) and (ii) to identify the location of the released nucleic acid in the biological sample, thereby identifying the location of the protein-nucleic acid complex in the biological sample.

In some embodiments, the biological sample is disposed on the substrate comprising the plurality of capture moieties. In some embodiments, the biological sample is disposed on a second substrate.

In some embodiments, the method further comprises aligning the second substrate comprising the biological sample with the first substrate comprising the plurality of capture moieties, such that at least a portion of the biological sample is aligned with at least a portion of the substrate comprising the plurality of capture moieties.

In some embodiments, aligning the biological sample with the first substrate comprising the plurality of capture moieties occurs after contacting the plurality of binding agents with the biological sample.

In some embodiments, the method further comprises permeabilizing the biological sample. In some embodiments, permeabilizing comprises use of one or more proteases. In some embodiments, the one or more proteases comprises pepsin, proteinase K, and collagenase.

In some embodiments, the method further comprises migrating the released nucleic acid to the array, and optionally, the migrating comprises electrophoresis.

In some embodiments, the method further comprises aligning a third substrate comprising the array with the substrate comprising the plurality of capture moieties, wherein the capture moiety is specifically bound to the binding agent of the binding agent-polypeptide-nucleic acid complex or the binding agent-protein-nucleic acid complex.

In some embodiments, when the capture moiety is specifically bound to the binding agent of the binding agent-polypeptide-nucleic acid complex or the binding agent-protein-nucleic acid complex, the method further comprises aligning the first substrate comprising the plurality of capture moieties with a third substrate comprising the array, such that at least a portion of the substrate comprising the plurality of capture moieties is aligned with at least a portion of the array,.

In some embodiments, the first substrate comprising the plurality of capture moieties comprises fiducial markers. In some embodiments, the third substrate comprising the array comprises fiducial markers.

In some embodiments, releasing the nucleic acid in step (d) comprises digesting the binding agent and polypeptide of the polypeptide-nucleic acid complex. In some embodiments, releasing the nucleic acid in step (d) comprises use of one or more enzymes after step (a), optionally wherein the one or more enzymes digests the binding agent and/or the polypeptide of the binding agent-polypeptide-nucleic acid complex or the binding agent and/or the protein of the binding agent-protein-nucleic acid complex. In some embodiments, releasing the nucleic acid in step (d) comprises use of one or more enzymes after step (b), optionally wherein the one or more enzymes digests the binding agent and/or the polypeptide of the binding agent-polypeptide-nucleic acid complex or the binding agent and/or the protein of the binding agent-protein-nucleic acid complex. In some embodiments, the one or more enzymes comprise pepsin or proteinase K.

In some embodiments, the capture domain of the capture probe comprises a poly(T) sequence. In some embodiments, the capture domain of the capture probe comprises a fixed sequence. In some embodiments, the capture probe is extended using the released nucleic acid as a template, thereby generating an extended capture probe. In some embodiments, the released nucleic acid is extended using the capture probe as a template.

In some embodiments, the determining step comprises sequencing. In some embodiments, the sequencing comprises high-throughput sequencing.

In some embodiments, the biological sample is fixed. In some embodiments, the biological sample is methanol-fixed, acetone-fixed, paraformaldehyde-fixed, or is formalin-fixed and paraffin-embedded (FFPE).

In some embodiments, the method further comprises staining the biological sample. In some embodiments, the staining comprises use of immunofluorescence, immunohistochemistry, or hematoxylin and/or eosin.

In some embodiments, the method further comprises imaging the biological sample.

In some embodiments, the biological sample is a tissue sample. In some embodiments, the tissue sample is a fixed tissue sample.

In some embodiments, the fixed tissue sample is a methanol-fixed tissue sample, an acetone-fixed tissue sample, a paraformaldehyde tissue sample, or a formalin-fixed paraffin-embedded tissue sample. In some embodiments, the tissue sample is a fresh-frozen tissue sample. In some embodiments, the biological sample is a tissue section. In some embodiments, the tissue section is a fixed tissue section. In some embodiments, the fixed tissue section is a methanol-fixed tissue section, an acetone-fixed tissue section, a paraformaldehyde tissue section, or a formalin-fixed paraffin-embedded tissue section. In some embodiments, the tissue section is a fresh-frozen tissue section.

In some embodiments, the polypeptide-nucleic acid complex comprises two or more polypeptides. In some embodiments, the polypeptide-nucleic acid complex comprises one polypeptide. In some embodiments, the protein-nucleic acid complex comprises two or more proteins. In some embodiments, the protein-nucleic acid complex comprises one protein.

In some embodiments, the polypeptide or protein comprises one or more of a ribosomal protein, a ribozyme, a histone, a transcription factor, a DNA-binding protein, and an RNA-binding protein. In some embodiments, the polypeptide-nucleic acid complex or the protein-nucleic acid complex comprises a ribonucleoprotein.

In some embodiments, the released nucleic acid comprises RNA. In some embodiments, the RNA is an mRNA. In some embodiments, the released nucleic acid comprises DNA. In some embodiments, the DNA is genomic DNA.

In some embodiments, the method further comprises incorporating a capture sequence onto an end of the nucleic acid of the binding agent-polypeptide-nucleic acid complex or the nucleic acid of the binding agent-protein-nucleic acid complex. In some embodiments, incorporating the capture sequence onto the end of the nucleic acid of the binding agent-polypeptide-nucleic acid complex or the nucleic acid of the binding agent-protein-nucleic acid complex comprises incorporating a poly(A) oligonucleotide onto the end of the nucleic acid of the binding agent-polypeptide-nucleic acid complex or the nucleic acid of the binding agent-protein-nucleic acid complex, optionally wherein the incorporating comprises ligating. In some embodiments, incorporating the capture sequence onto the end of the nucleic acid of the binding agent-polypeptide-nucleic acid complex or the nucleic acid of the binding agent-protein-nucleic acid complex comprises the use of a ligase, a poly(A) polymerase, a terminal transferase, a plurality of dATPs, or a combination thereof.

In some embodiments, the binding moiety comprises biotin. In some embodiments, the capture moiety comprises avidin or streptavidin.

In some embodiments, the capture probe further comprises one or more functional domains, a cleavage domain, a unique molecular identifier, and combinations thereof. In some embodiments, the one or more functional domains comprises a primer binding site or a sequencing specific site.

Also provided herein are methods of determining a location of a polypeptide-nucleic acid complex in a biological sample, the method comprising: (a) aligning the biological sample with a substrate comprising: (i) a plurality of capture probes, wherein a capture probe of the plurality of capture probes comprises: (i) a spatial barcode and (ii) a capture domain; and (ii) a plurality of capture moieties, wherein a capture moiety specifically binds a binding moiety; (b) contacting a plurality of binding agents with the biological sample, wherein a binding agent of the plurality of binding agents specifically binds a polypeptide of the polypeptide-nucleic acid complex thereby forming a binding agent-polypeptide-nucleic acid complex; (c) binding the binding agent of the binding agent-polypeptide-nucleic acid complex to the capture moiety of the plurality of capture moieties; (d) releasing the nucleic acid of the binding agent-polypeptide-nucleic acid complex, thereby generating a released nucleic acid; (e) hybridizing the released nucleic acid to the capture domain of the capture probe; and (f) determining (i) the sequence of the spatial barcode, or a complement thereof, and (ii) all or a portion of the sequence of the released nucleic acid, or a complement thereof, and using the determined sequences of (i) and (ii) to determine the location of the released nucleic acid in the biological sample, thereby determining the location of the polypeptide-nucleic acid complex in the biological sample.

Also provided herein are kits comprising: (a) an array comprising a plurality of capture probes, wherein a capture probe of the plurality of capture probes comprises: (i) a spatial barcode and (ii) a capture domain; (b) a first substrate comprising a plurality of capture moieties, wherein a capture moiety of the plurality of capture moieties specifically binds a binding moiety; (c) a plurality of binding agents, wherein a binding agent specifically binds a polypeptide of a polypeptide-nucleic acid complex and comprises the binding moiety; and (d) instructions for performing any of the methods described herein (e.g., methods comprising an array and a first substrate comprising a plurality of capture moieties).

Also provided herein are kits comprising: (a) an array comprising: (i) a plurality of capture probes, wherein a capture probe of the plurality of capture probes comprises: (i) a spatial barcode and (ii) a capture domain; (ii) a plurality of capture moieties, wherein a capture moiety of the plurality of capture moieties specifically binds a binding agent; (b) a plurality of binding agents, wherein a binding agent specifically binds a polypeptide of a polypeptide-nucleic acid complex and comprises the binding moiety; and (c) instructions for performing any of the methods described herein (e.g., methods comprising an array comprising a plurality of capture probes and a plurality of capture moieties).

In some embodiments, the kit further comprises one or more permeabilization reagents. In some embodiments, the one or more permeabilization reagents comprises one or more proteases, a DNase, an RNase, a lipase, a detergent, and combinations thereof. In some embodiments, the one or more proteases comprise pepsin, proteinase K, and collagenase.

In some embodiments, the capture probe further comprises one or more functional domains, a cleavage domain, a unique molecular identifier, and combinations thereof. In some embodiments, the one or more functional domains comprises a primer binding site or a sequencing specific site.

In some embodiments, the kit further comprises a polymerase. In some embodiments, the polymerase comprises a reverse transcriptase and/or a DNA polymerase.

Also provided herein are compositions comprising: (a) an array comprising a plurality of capture probes, wherein a capture probe of the plurality of capture probes comprises: (i) a spatial barcode and (ii) a capture domain; and (b) a substrate comprising a plurality of capture moieties, wherein a capture moiety of the plurality of capture moieties is specifically bound to a binding moiety of a binding agent-polypeptide-nucleic acid complex.

Also provided herein are compositions comprising: (a) an array comprising: (i) a plurality of capture probes, wherein a capture probe of the plurality of capture probes comprises: (i) a spatial barcode and (ii) a capture domain; and (ii) a plurality of capture moieties, wherein a capture moiety of the plurality of capture moieties is specifically bound to a binding moiety of a binding agent-polypeptide-nucleic acid complex.

Also provided herein are compositions comprising: (a) an array comprising a plurality of capture probes, wherein a capture probe of the plurality of capture probes comprises: (i) a spatial barcode and (ii) a capture domain, wherein the capture domain is hybridized to a released nucleic acid of a binding agent-polypeptide-nucleic acid complex; and (b) a substrate comprising a plurality of capture moieties, wherein a capture moiety of the plurality of capture moieties is specifically bound to a binding moiety of a binding agent-polypeptide complex.

Also provided herein are compositions comprising: an array comprising: (i) a plurality of capture probes, wherein a capture probe of the plurality of capture probes comprises: (i) a spatial barcode and (ii) a capture domain, wherein the capture domain is hybridized to a released nucleic acid of a binding agent-polypeptide-nucleic acid complex; and (ii) a plurality of capture moieties, wherein a capture moiety of the plurality of capture moieties is specifically bound to a binding moiety of a binding agent-polypeptide complex.

In some embodiments, the composition further comprises one or more permeabilization reagents. In some embodiments, the one or more permeabilization reagents comprises one or more proteases, a DNase, an RNase, a lipase, a detergent, and combinations thereof. In some embodiments, the one or more proteases comprise pepsin, proteinase K, and collagenase.

In some embodiments, the capture probe further comprises one or more functional domains, a cleavage domain, a unique molecular identifier (UMI), and combinations thereof. In some embodiments, the one or more functional domains comprises a primer binding site or a sequencing specific site.

In some embodiments, the composition further comprises a polymerase. In some embodiments, the polymerase comprises a reverse transcriptase and/or a DNA polymerase.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows and exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

### DETAILED DESCRIPTION

### Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I) (b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I) (c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology and biomolecules. PAXgene provides a two-reagent fixative system in which tissue is firstly fixed in a solution containing methanol and acetic acid, then stabilized in a solution containing ethanol. See, Ergin B. et al., J Proteome Res. 2010 Oct. 1;9(10):5188-96; Kap M. et al., PLOS One.; 6(11):e27704 (2011); and Mathieson W. et al., Am J Clin Pathol.; 146(1):25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, PAXgene, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I) (d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the array of capture probes is a second substrate. In this configuration, one or more analytes or analyte derivatives (e.g., intermediate agents; e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference.

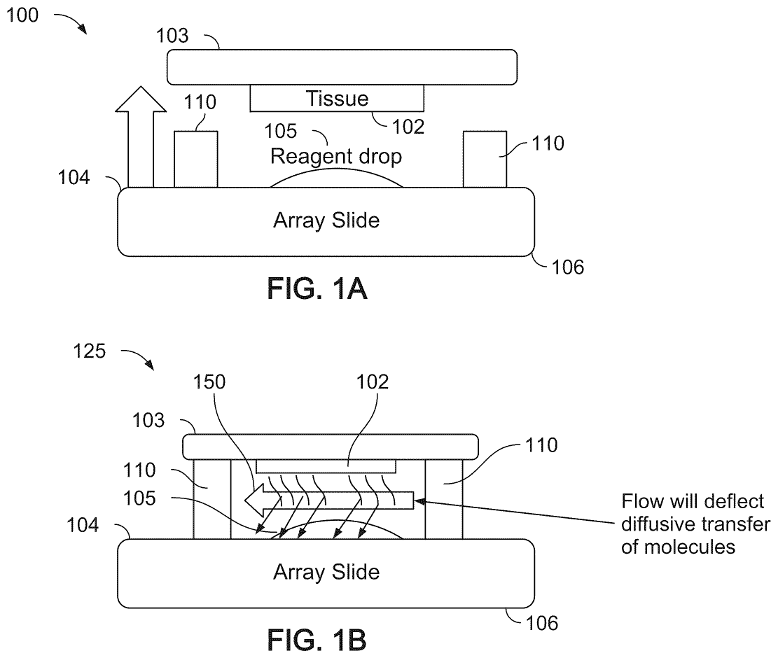

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

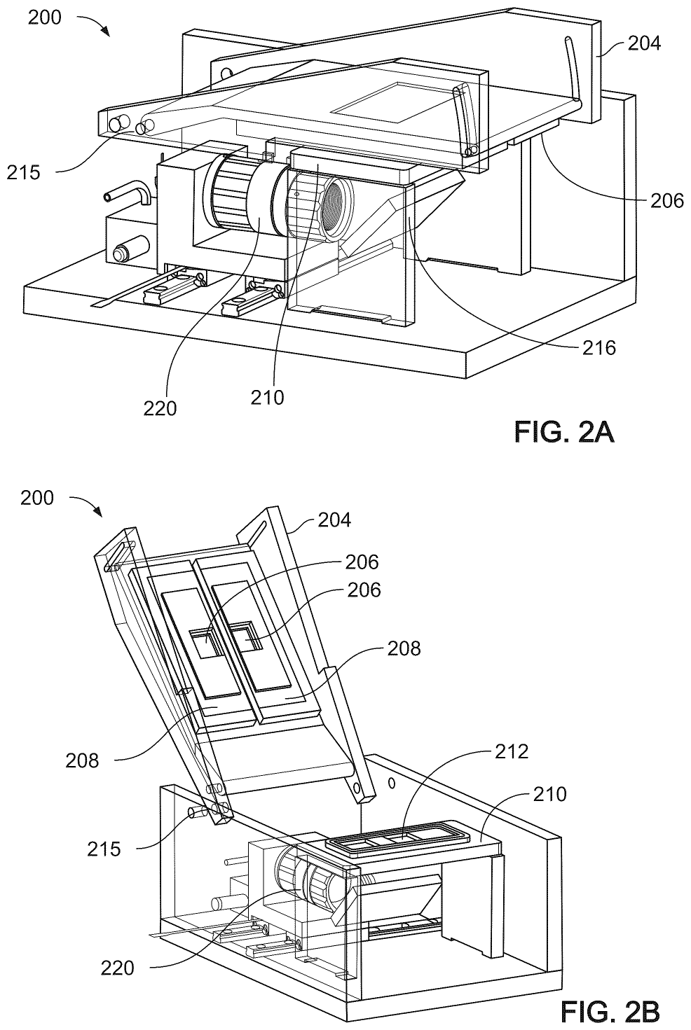

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

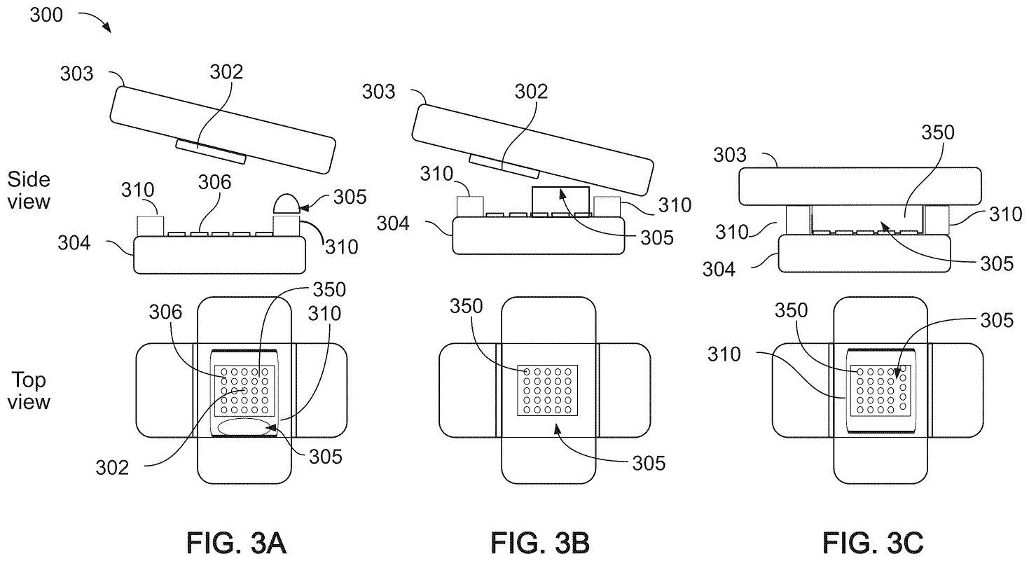

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

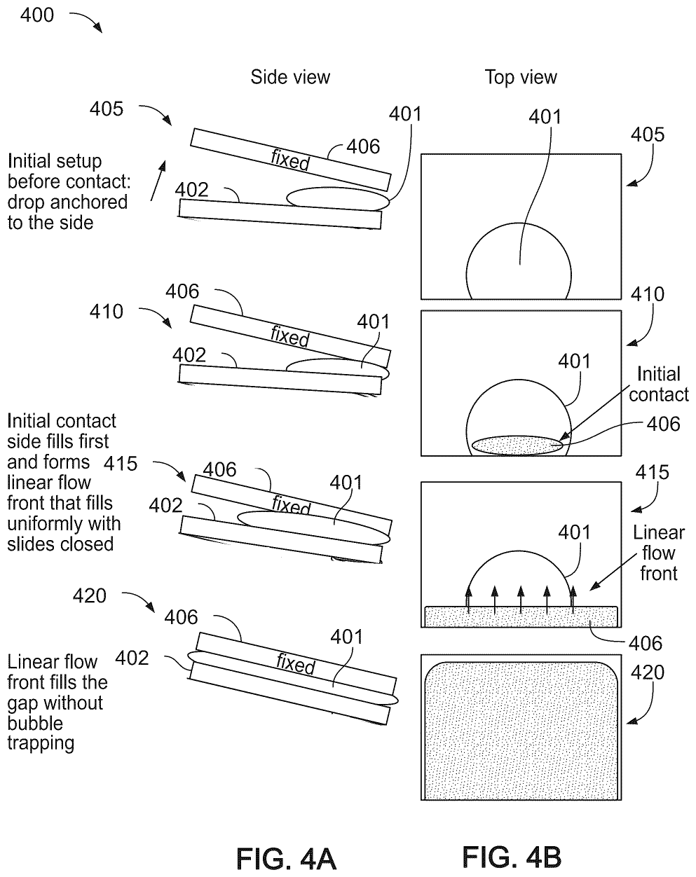

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™,SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II) (a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663, which is herein incorporated by reference. Some quality control measures are described in Section (II)(h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II) (c) of PCT Publication No. WO2020/176788 and/or U.S. Patent Application Publication No. 2020/0277663, which is herein incorporated by reference. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II) (e) of PCT Publication No. WO2020/176788 and/or U.S. Patent Application Publication No. 2020/0277663, which is herein incorporated by reference.

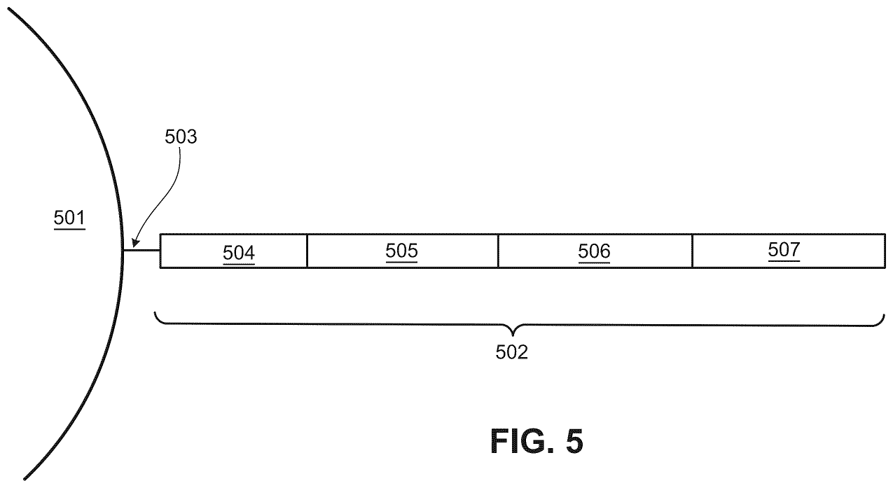

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

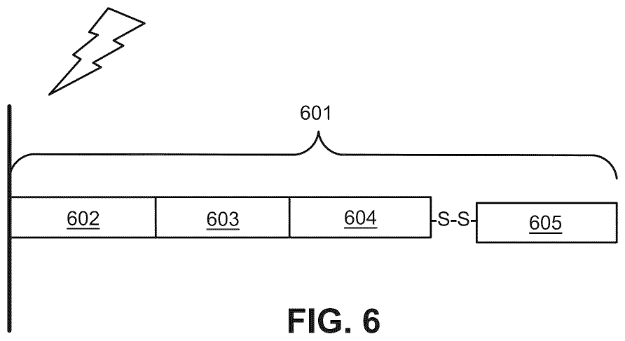

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

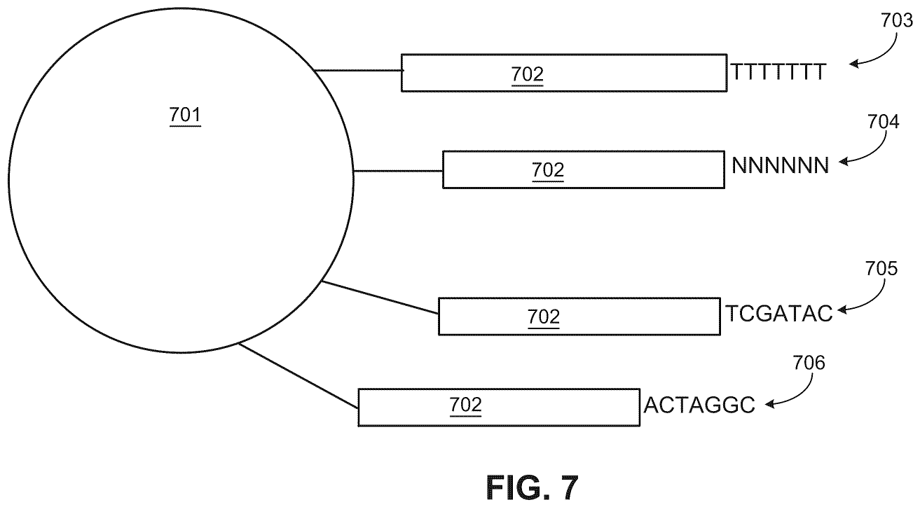

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

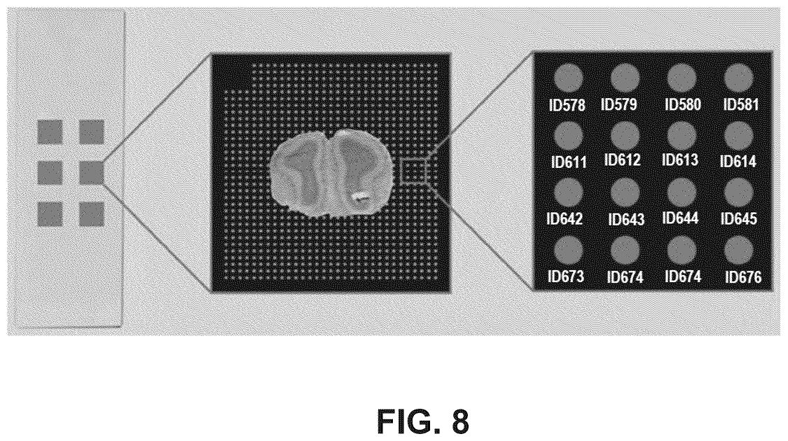

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578,ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

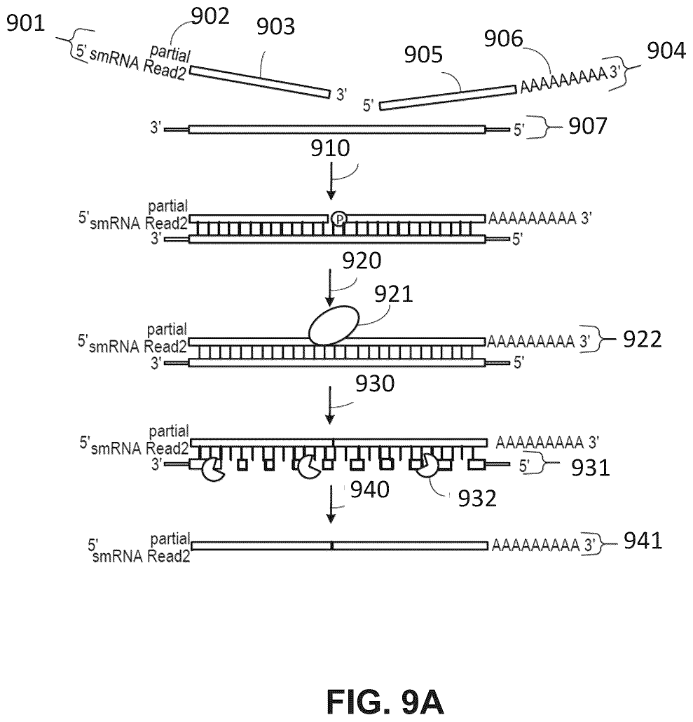

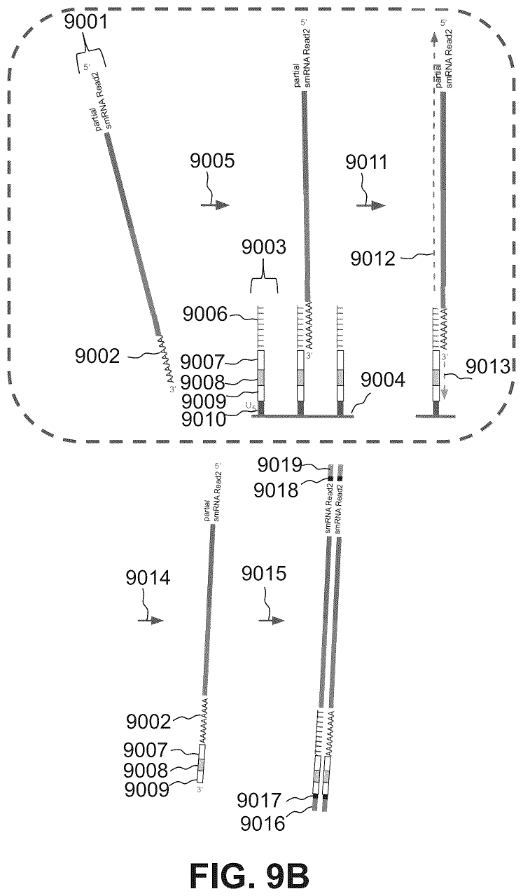

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Publication. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the polymerization reagents can be used to extend the capture probes 9011 to produce spatially-barcoded full-length cDNA 9012 and 9013 from the captured ligation products (e.g., ligation products). The ligation products can be extended using the capture probe as a template to include a complement of the capture probe, thereby generating extended ligation products.

In some embodiments, the extended ligation products can be denatured 9014, released from the capture probe, and transferred (e.g., to a clean tube) for amplification and/or library construction. The spatially-barcoded ligation products can be amplified 9015 via PCR prior to library construction. P5 9016, i5 9017, i7 9018, and P7 9019 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663, which is herein incorporated by reference.

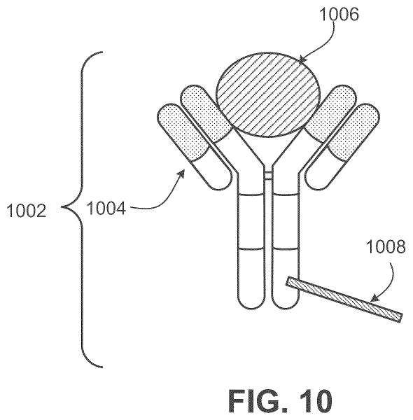

FIG. 10 is a schematic diagram of an exemplary analyte capture agent 1002 comprised of an analyte binding moiety 1004 and an analyte-binding moiety barcode domain 1008. The exemplary analyte binding moiety 1004 is capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

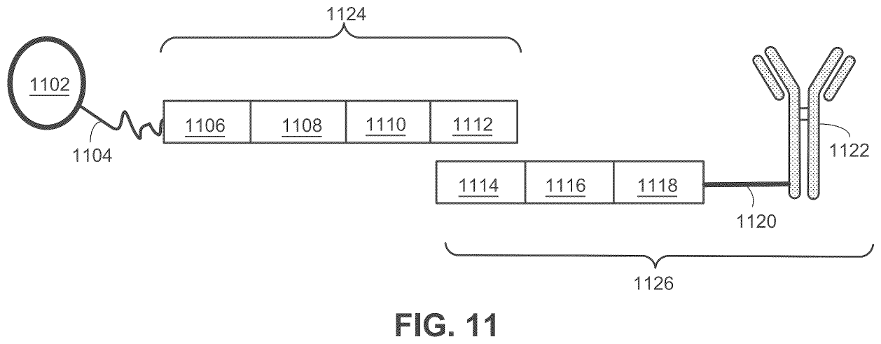

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable, thermal-cleavable, or an enzyme cleavable linker. In some instances, the cleavable linker is a disulfide linker. A disulfide linker can be cleaved by use of a reducing agent, such as dithiothreitol (DTT), beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II) (e) (ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a sealable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Spatial Detection of Nucleic Acids that Interact with Polypeptides

This disclosure features methods, compositions, and kits of isolating one or more polypeptides (or one or more proteins) that interact with a nucleic acid to determine the location of the polypeptide-nucleic acid complex (or protein-nucleic acid complex) in a biological sample. For example, the methods described herein can utilize: (i) binding agents to isolate a polypeptide-nucleic acid complex or a protein-nucleic acid complex on a substrate that includes a plurality of capture moieties and (ii) a spatial array to determine the location of the nucleic acid from the polypeptide-nucleic acid complex or protein-nucleic acid complex.

Thus, provided herein are methods of determining a location of a polypeptide-nucleic acid complex or a protein-nucleic acid complex in a biological sample. In some embodiments, the methods include forming a binding agent-polypeptide-nucleic acid complex or binding agent-protein-nucleic acid complex in a biological sample, binding the binding agent-polypeptide-nucleic acid complex or binding agent-protein-nucleic acid complex to a capture moiety on a substrate that includes a plurality of capture moieties, releasing the nucleic acid of the polypeptide-nucleic acid complex or protein-nucleic acid complex, and hybridizing the released nucleic acid to a capture probe on an array that includes a plurality of capture probes. Upon interaction of the released nucleic acid with the array having a plurality of capture probes, the location and/or abundance of the polypeptide-nucleic acid complex or protein-nucleic acid complex in the biological sample can be determined, as provided herein.

Also provided herein are methods of determining a location of a polypeptide-nucleic acid complex in a biological sample, comprising: (a) contacting a plurality of binding agents with the biological sample, wherein a binding agent of the plurality of binding agents binds a polypeptide of the polypeptide-nucleic acid complex in the biological sample thereby forming a binding agent-polypeptide-nucleic acid complex and wherein the binding agent comprises a binding moiety; (b) aligning the biological sample with a first substrate comprising a plurality of capture moieties such that at least a portion of the biological sample is aligned with at least a portion of the first substrate comprising the plurality of capture moieties; (c) contacting the binding moiety of the binding agent of the binding agent-polypeptide-nucleic acid complex with a capture moiety of the plurality of capture moieties, such that the binding agent-polypeptide-nucleic acid complex is captured on the first substrate; (d) releasing the nucleic acid of the binding agent-polypeptide-nucleic acid complex captured on the first substrate, thereby generating a released nucleic acid; (e) hybridizing the released nucleic acid to a capture domain of a capture probe comprised in an array comprising a plurality of capture probes, wherein the capture probe of the plurality of capture probes comprises: (i) a spatial barcode, and (ii) the capture domain; and (f) determining (i) the sequence of the spatial barcode, or a complement thereof, and (ii) all or a portion of the sequence of the released nucleic acid, or a complement thereof, and using the determined sequences of (i) and (ii) to determine the location of the released nucleic acid in the biological sample, thereby determining the location of the polypeptide-nucleic acid complex in the biological sample.

Also provided herein are methods of determining a location of a polypeptide-nucleic acid complex in a biological sample, the methods including (a) contacting a plurality of binding agents with the biological sample, wherein a binding agent of the plurality of binding agents specifically binds a polypeptide of the polypeptide-nucleic acid complex thereby forming a binding agent-polypeptide-nucleic acid complex and wherein the binding agent comprises a binding moiety; (b) aligning the biological sample with a first substrate comprising a plurality of capture moieties, wherein the binding moiety of the binding agent interacts with a capture moiety of the plurality of capture moieties; (c) providing an array comprising a plurality of capture probes, wherein a capture probe of the plurality of capture probes comprises: (i) a spatial barcode, and (ii) a capture domain; (d) releasing the nucleic acid of the binding agent-polypeptide-nucleic acid complex, thereby generating a released nucleic acid; (e) hybridizing the released nucleic acid to the capture domain of the capture probe; and (f) determining (i) the sequence of the spatial barcode, or a complement thereof, and (ii) all or a portion of the sequence of the released nucleic acid, or a complement thereof, and using the determined sequences of (i) and (ii) to determine the location of the released nucleic acid in the biological sample, thereby determining the location of the polypeptide-nucleic acid complex in the biological sample.

Also provided herein are methods of determining a location of a protein-nucleic acid complex in a biological sample, the methods including (a) contacting a plurality of binding agents with the biological sample, wherein a binding agent of the plurality of binding agents specifically binds a protein of the protein-nucleic acid complex thereby forming a binding agent-protein-nucleic acid complex and wherein the binding agent comprises a binding moiety; (b) aligning the biological sample with a first substrate comprising a plurality of capture moieties, wherein the binding moiety of the binding agent interacts with a capture moiety of the plurality of capture moieties; (c) providing an array comprising a plurality of capture probes, wherein a capture probe of the plurality of capture probes comprises: (i) a spatial barcode, and (ii) a capture domain; (d) releasing the nucleic acid of the binding agent-protein-nucleic acid complex, thereby generating a released nucleic acid; (e) hybridizing the released nucleic acid to the capture domain of the capture probe; and (f) determining (i) the sequence of the spatial barcode, or a complement thereof, and (ii) all or a portion of the sequence of the released nucleic acid, or a complement thereof, and using the determined sequences of (i) and (ii) to determine the location of the released nucleic acid in the biological sample, thereby determining the location of the protein-nucleic acid complex in the biological sample.

Also provided herein are methods of determining a location of a polypeptide-nucleic acid complex in a biological sample, the method comprising: (a) aligning the biological sample with a substrate comprising: (i) a plurality of capture probes, wherein a capture probe of the plurality of capture probes comprises: (i) a spatial barcode and (ii) a capture domain; and (ii) a plurality of capture moieties, wherein a capture moiety (e.g., any of the capture moieties described herein) specifically binds a binding moiety (e.g., any of the binding moieties described herein); (b) contacting a plurality of binding agents with the biological sample, wherein a binding agent of the plurality of binding agents specifically binds a polypeptide of the polypeptide-nucleic acid complex thereby forming a binding agent-polypeptide-nucleic acid complex; (c) binding the binding agent of the binding agent-polypeptide-nucleic acid complex to the capture moiety of the plurality of capture moieties; (d) releasing the nucleic acid of the binding agent-polypeptide-nucleic acid complex, thereby generating a released nucleic acid; (e) hybridizing the released nucleic acid to the capture domain of the capture probe; and (f) determining (i) the sequence of the spatial barcode, or a complement thereof, and (ii) all or a portion of the sequence of the released nucleic acid, or a complement thereof, and using the determined sequences of (i) and (ii) to determine the location of the released nucleic acid in the biological sample, thereby determining the location of the polypeptide-nucleic acid complex in the biological sample.

In some embodiments, the plurality of binding agents includes different types or species of binding agents. In some embodiments, the plurality of binding agents includes one type or species of binding agent. In some embodiments, the plurality of capture moieties includes different types or species of capture moieties. In some embodiments, the plurality of capture moieties includes one type or species of capture moiety.

The term “specifically binds,” or the like, means that a protein and/or polypeptide or a fragment thereof (e.g., a binding agent or binding moiety as described herein) forms a complex with another molecule (e.g., a capture moiety as described herein) that is relatively stable under physiologic conditions. In embodiments, “specifically binds,” means that a first molecule (e.g., a binding agent) preferably or exclusively forms a complex with a second molecule (e.g., a target polypeptide or protein) over other molecules (e.g., non-target polypeptides or proteins) which may be present. For example, an anti-Her2 antibody may specifically bind to Her2 as compared to other cell surface receptors, such that the anti-Her2 antibody exhibits higher affinity for Her2compared to other cell surface receptors (e.g., IR, LHR). In some embodiments, the k_{off} for a complex in which the members are specifically bound is smaller than the k_{off} for a complex in which the members are not specifically bound. Methods for determining whether a protein or polypeptide (e.g., a binding agent as described herein) specifically binds to another molecule (e.g., a capture moiety as described herein) are well known in the art and include, for example, equilibrium dialysis, surface plasmon resonance, and the like. For example, a binding agent that “specifically binds” to a capture moiety, as used in the context of the present disclosure, can include proteins or polypeptides that bind a capture moiety (e.g., any of the capture moieties described herein) or portion thereof with a Ka of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM.

The methods described herein can be performed on any type of biological sample. In some embodiments, the biological sample is a fresh tissue sample. In some embodiments, the biological sample is a frozen sample. In some embodiments, the biological sample was previously frozen. In some embodiments, the biological sample is a fresh-frozen tissue sample. In some embodiments, the biological sample is a formalin-fixed, paraffin embedded (FFPE) sample.

In some embodiments, the biological sample can be fixed and/or stained. In some embodiments, the biological sample is stained after fixation. In some embodiments, the biological sample is stained before fixation. In some embodiments, the staining includes optical labels as described herein, including, but not limited to, fluorescent (e.g., fluorophore), radioactive (e.g., radioisotope), chemiluminescent (e.g., a chemiluminescent compound), a bioluminescent compound, calorimetric, or colorimetric detectable labels. In some embodiments, the staining includes a fluorescent antibody directed to a target analyte (e.g., cell surface or intracellular proteins) in the biological sample. In some embodiments, the staining includes an immunohistochemistry stain directed to a target analyte (e.g., cell surface or intracellular proteins) in the biological sample. In some embodiments, the staining includes a chemical stain, such as hematoxylin and eosin (H&E) or Periodic acid-Schiff (PAS). In some embodiments, staining the biological sample includes the use of a biological stain including, but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, significant time (e.g., days, months, or years) can elapse between staining and/or imaging the biological sample.

In some embodiments, the biological sample is imaged. In some embodiments, the biological sample is imaged after fixation. In some embodiments, the biological sample is imaged before fixation. In some embodiments, imaging includes one or more of expansion microscopy, bright field microscopy, dark field microscopy, phase contrast microscopy, electron microscopy, fluorescence microscopy, reflection microscopy, interference microscopy and confocal microscopy.

In some embodiments, the biological sample is permeabilized. Permeabilization of a biological sample can occur on a substrate (e.g., a second substrate as described herein). In some embodiments, the biological sample is permeabilized prior to aligning the biological sample with the first substrate comprising a plurality of capture moieties. In some embodiments, the biological sample is permeabilized with a protease and/or one or more detergents. In some embodiments, the protease is one or more of pepsin, Proteinase K, and collagenase.

In some embodiments, the biological sample is disposed on the first substrate having the plurality of capture moieties. In some embodiments, aligning the biological sample with the first substrate having the plurality of capture moieties occurs after contacting the plurality of binding agents with the biological sample.

In some embodiments, the biological sample is disposed on a second substrate. In some embodiments, the method further comprises aligning the second substrate having the biological sample with the first substrate having the plurality of capture moieties, such that at least a portion of the biological sample is aligned with at least a portion of the first substrate comprising the plurality of capture moieties. In some embodiments, aligning the second substrate comprising the biological sample with the first substrate comprising the plurality of capture moieties occurs after contacting the plurality of binding agents with the biological sample.

As used herein, “sandwiching” or a “sandwiching process” refers to aligning a second substrate comprising the biological sample with a first substrate comprising the plurality of capture moieties and/or aligning a substrate (e.g., a third substrate) comprising an array of capture probes with a first substrate comprising the plurality of capture moieties. The methods described herein can include multiple sandwiching steps.

In some embodiments, the second substrate comprising the biological sample is aligned with the first substrate comprising the plurality of capture moieties in a sandwich configuration (e.g., a sandwich configuration as described herein). For example, a first substrate having a plurality of capture moieties and a second substrate including a biological sample can be brought into proximity with one another. In some embodiments, the first substrate is aligned with the second substrate, such that at least a portion of the biological sample is aligned with at least a portion of the capture moieties (e.g., aligned in a sandwich configuration). A reagent medium within a gap between the first substrate having the plurality of capture moieties and the second substrate having the biological sample can create a liquid interface between the two substrates. In some embodiments, a binding agent-polypeptide-nucleic acid complex or binding agent-protein-nucleic acid complex in the biological sample may release from the biological sample, and actively or passively migrate (e.g., diffuse) across the gap toward the capture moieties on the first substrate. Alternatively, in certain embodiments, migration of the binding agent-polypeptide-nucleic acid complex or binding agent-protein-nucleic acid complex from the biological sample is performed actively (e.g., electrophoretic, by applying an electric field to promote migration). In certain embodiments, a binding moiety of the binding agent in the binding agent-polypeptide-nucleic acid complex (or binding agent-protein-nucleic acid complex) contacts the capture moiety such that the binding agent-polypeptide-nucleic acid complex (or binding agent-protein-nucleic acid complex) is captured on the substrate comprising the plurality of capture moieties.

A capture sequence can be incorporated (e.g., added) onto an end of a nucleic acid (e.g., the nucleic acid of the binding agent-polypeptide-nucleic acid complex or the binding agent-protein-nucleic acid complex). Various methods are known to add a capture sequence (e.g., a sequence capable of hybridizing to a capture domain of a capture probe on a spatial array) to nucleic acids (e.g., the nucleic acid of the binding agent-polypeptide-nucleic acid complex or the binding agent-protein-nucleic acid complex). In some embodiments, incorporating the capture sequence onto an end of the nucleic acid of the binding agent-polypeptide-nucleic acid complex or the binding agent-protein-nucleic acid complex includes ligating a poly(A) oligonucleotide onto an end of the nucleic acid of the binding agent-polypeptide-nucleic acid complex or the binding agent-protein-nucleic acid complex. For example, a plurality of poly(A) oligonucleotides and a ligase (e.g., any of the ligases provided herein) can be contacted with the biological sample or the first substrate (e.g., a first substrate having a plurality of capture moieties, wherein a capture moiety is specifically bound to a binding agent-polypeptide-nucleic acid complex or a binding agent-protein-nucleic acid complex). The ligase enzyme ligates a poly(A) oligonucleotide onto the ends of the nucleic acid. The poly(A) oligonucleotide can be about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or more nucleotides long. The poly(A) oligonucleotide only needs to be sufficiently long (e.g., about 5 nucleotides) to hybridize the capture domain (e.g., a poly(T) sequence) of a capture probe.

Alternatively, a capture sequence (e.g., a sequence substantially complementary to the capture domain of a capture probe) can be added onto a nucleic acid (e.g., the nucleic acid of the binding agent-polypeptide-nucleic acid complex or the binding agent-protein-nucleic acid complex) with a terminal transferase and a plurality of dATPs or a poly(A) polymerase and a plurality of dATPs. In some embodiments, the terminal transferase is terminal deoxynucleotidyl transferase. Terminal transferases are able to add nucleotides (e.g., dATPs) in a template-independent manner. In some embodiments, a terminal transferase adds one or more dATPs (e.g., about 5, about 6, about 7, about 8, about 9, about 10, about 11, about 12, about 13, about 14, about 15, about 16, about 17, about 18, about 19, about 20, about 21, about 22, about 23, about 24, about 25 dATPs) onto the ends of the nucleic acid, thereby generating a poly(A) sequence. The generated poly(A) sequence only needs to be sufficiently long (e.g., about 5 or more nucleotides) to hybridize the capture domain (e.g., a poly(T) sequence) of a capture probe.

The first substrate comprising the plurality of capture moieties can be aligned to any of the variety of substrates, including substrates with arrays, described herein. In some embodiments, a method described herein further comprises aligning a substrate (e.g., a third substrate) comprising the array (e.g., any of the arrays described herein) with the first substrate comprising the plurality of capture moieties, wherein the capture moiety is specifically bound to the binding agent of the binding agent-polypeptide-nucleic acid complex or the binding agent-protein-nucleic acid complex. In some embodiments, when first substrate includes the capture moiety bound to the binding agent of the binding agent-polypeptide-nucleic acid complex or the binding agent-protein-nucleic acid complex, the method further comprises aligning the first substrate comprising the plurality of capture moieties or binding agent-polypeptide-nucleic acid complex captured thereon with a substrate (e.g., a third substrate) comprising the array, such that at least a portion of the first substrate is aligned with at least a portion of the array.

In some embodiments, the array comprising the plurality of capture probes is aligned with the first substrate comprising the plurality of capture moieties (e.g., a first substrate having a capture moiety that is specifically bound to the binding agent of a binding agent-polypeptide-nucleic acid complex or a binding agent-protein-nucleic acid complex) in a sandwich configuration (e.g., a sandwich configuration as described herein). For example, a first substrate having a plurality of capture moieties (e.g., a first substrate having a capture moiety that is specifically bound to the binding agent of a binding agent-polypeptide-nucleic acid complex or a binding agent-protein-nucleic acid complex) and an array comprising the plurality of capture probes can be brought into proximity or contact with one another. In some embodiments, the first substrate is aligned with a substrate (e.g., a third substrate) comprising the array, such that at least a portion of the capture moieties is aligned with at least a portion of the capture probes (e.g., aligned in a sandwich configuration). A reagent medium within a gap between the first substrate having the plurality of capture moieties and the substrate (e.g., a third substrate) including the array of capture probes can create a liquid interface between the two substrates. In some embodiments, a nucleic acid of the binding agent-polypeptide-nucleic acid complex or binding agent-protein-nucleic acid complex that is specifically bound to a capture moiety of the first substrate may release from the binding agent-polypeptide-nucleic acid complex or binding agent-protein-nucleic acid complex, and actively or passively migrate (e.g., diffuse) across the gap toward the capture probes on the array. In certain embodiments, migration of the binding agent-polypeptide-nucleic acid complex or binding agent-protein-nucleic acid complex from the biological sample is performed actively (e.g., electrophoretic, by applying an electric field to promote migration). In some embodiments of the sandwich configuration, there is no gap between the aligned substrates, e.g., a first substrate having a binding agent-polypeptide-nucleic acid complex or a binding agent-protein-nucleic acid complex captured thereon and an array (e.g., disposed on a third substrate) comprising the plurality of capture probes can be brought into proximity or contact with one another.

In some embodiments, the method further comprises migrating the released nucleic acid to the array (e.g., an array on a third substrate). In some embodiments, the migrating comprises electrophoresis.

In some embodiments, the first substrate comprising the plurality of capture moieties comprises fiducial markers. In some embodiments, the substrate (e.g., a third substrate) comprising the array comprises fiducial markers.

A capture probe of the plurality of capture probes on an array can be any of the variety of capture probes described herein. In some embodiments, a capture probe of the plurality of capture probes includes, in a 5′ to a 3′ direction, a spatial barcode and a capture domain. In some embodiments, the capture domain hybridizes to a capture sequence. In some embodiments, the capture domain is a poly(T) capture domain. In some embodiments, the capture domain is not a poly(T) sequence. In some embodiments, the capture domain is a fixed sequence. As used herein, a “fixed sequence” is a non-random sequence. In some embodiments, the capture domain is a gene-specific sequence.

In some embodiments, a capture probe can include one or more functional domains, and/or a cleavage domain. A functional domain typically includes a functional nucleotide sequence for a downstream analytical step in the overall analysis procedure. In some embodiments, the functional domain can include a sequencing domain. In some embodiments, the functional domain can include an amplification (e.g., PCR) domain. In some embodiments, the functional domain can include a primer binding sequence. In some embodiments, a capture probe can include a unique molecular identifier as described herein. In some embodiments, the unique molecular identifier is located 5′ to the capture domain in the capture probe.

The nucleic acid from the binding agent-polypeptide-nucleic acid complex or the binding agent-protein-nucleic acid complex can be released from the binding agent-polypeptide-nucleic acid complex or binding agent-polypeptide-nucleic acid complex via, e.g., digestion of the binding agent and/or polypeptide or protein. In some embodiments, releasing the nucleic acid comprises use of one or more enzymes. For example, the one or more enzymes can digest the binding agent and/or the polypeptide of the binding agent-polypeptide-nucleic acid complex or the binding agent and/or the protein of the binding agent-protein-nucleic acid complex. In some embodiments, the one or more enzymes comprise pepsin or proteinase K. In some embodiments, releasing the nucleic acid comprises use of one or more enzymes after step (a). In some embodiments, releasing the nucleic acid comprises use of one or more enzymes after step (b).

Upon hybridization of the released nucleic acid to the capture domain of the capture probe (e.g., any of the capture probes described herein), the capture probe can be extended using the released nucleic acid as a template and/or the released nucleic acid can be extended using the capture probe as a template. In some embodiments, extending the released nucleic acid includes reverse transcription e.g., using a reverse transcriptase. In some embodiments, extending the released nucleic acid and/or capture probe involves use of a polymerase. The extended nucleic acid and/or extended capture probe can be harvested from the array, sequencing libraries generated and sequenced. The sequencing of the released nucleic acid can be used to determine the location of the polypeptide-nucleic acid complex or the protein-nucleic acid complex in the biological sample.

In some embodiments, the released nucleic acid or complement thereof can be prepared for downstream applications, such as generation of a sequencing library and next-generation sequencing. Generating sequencing libraries are known in the art. For example, the released nucleic acid or complement thereof can be purified and collected for downstream amplification steps. The amplification products can be amplified using PCR, where primer binding sites flank the spatial barcode and target nucleic acid, or a complement thereof, generating a library associated with a particular spatial barcode. In some embodiments, the library preparation can be quantitated and/or quality controlled to verify the success of the library preparation steps. The library amplicons are sequenced and analyzed to decode spatial information of the released nucleic acid.

Non-limiting examples of nucleic acids included in the polypeptide-nucleic acid complex or protein-nucleic acid complex include DNA and RNA. Non-limiting examples of DNA include genomic DNA, methylated DNA, fragmented DNA, mitochondrial DNA, in situ synthesized PCR products, and viral DNA.

Non-limiting examples of RNA include various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). The RNA can be from an RNA virus, for example RNA viruses from Group III, IV or V of the Baltimore classification system. The RNA can be from a retrovirus, such as a virus from Group VI of the Baltimore classification system.

A polypeptide-nucleic acid complex or protein-nucleic acid complex can include one, two, three, four or more polypeptides and/or proteins. For example, in some embodiments, the polypeptide-nucleic acid complex comprises two or more polypeptides. In some embodiments, the polypeptide-nucleic acid complex comprises one polypeptide. In some embodiments, the protein-nucleic acid complex comprises two or more proteins. In some embodiments, the protein-nucleic acid complex comprises one protein.

Non-limiting examples of a polypeptide or protein that interact with a nucleic acid include a ribosomal protein (e.g., ribosomal subunits such as, S3A, S2, S3, S4, S5 S6), a ribozyme, a DNA-binding protein (e.g., a transcription factor), and an RNA-binding protein.

In some embodiments, the polypeptide or protein is an RNA-binding protein. Non-limiting examples of an RNA-binding protein include RBM5, RBM6, YBX2, CSDE1, PTBP1, ZC3H3, MATR3, SAMD4A, YTHDC2, CUGBP2, PUM2, RC3H2, ZC3H11A, PARP12, CSDA, SFRS8, EIF4B, U2AF2, SFRS14, SPEN, ZC3H15, YBX1, ELAVL1, THUMPD1, DHX8, SRBD1, PABPC1, DAZAP1, IGF2BP2, ZNF638, SART3, MKRN2, RBM7, RBMS2, MBNL3, SNRPA, SYNJ2, A2BP1, DDX43, KIAA0020, CNOT4, YTHDC1, PPIE, CHERP, RBM22, KHSRP, FUS, RBM41, PCBP4, PABPC4, TNRC6A, RBM27, HNRNPC, DAZL, HNRNPH3, SETDIA, CIRBP, HNRNPM, TRMT2A, SF3A1, SNRPD3, POLDIP3, ZMAT5, RBM9, ZC3H7B, RBM23, SFRS5, ZC3H14, ACIN1, PABPN1, PABPCIL, BRUNOL4, CSTF2, ZC3H12B, FMR1, HTATSF1, RBM3, ESRP2, MTHFSD, DNAJC17, MYEF2, ESRP1, HNRNPL, SNRNP70, SFRS16, TRMT1, NOVA2, SF4, ZC3HAV1, EIF3B, RBM28, LSM5, EIF4H, ELAVL2, FUBP3, CPEB3, LARP4B, SUPT6H, PPARGC1A, CPSF6, KRR1, SFRS9, SFRS3, RBM24, KHDRBS2, QKI, CPEB4, FXR1, NCL, HDLBP, SFRS7, PNO1, TIA1, SFRS4, SFPQ, SFRS11, PRPF3, PTBP2, ROD1, RBM18, C14orf156, RBM25, ENOX1, TARDBP, AKAP1, PSPC1, ZCCHC17, KHDRBS1, ZC3H7A, HNRNPA2B1, BICC1, RBM19, ZC3H13, SFRS6, RNF113A, SNRPD2, SNRPB, SNRPB2, NRNPR, RALY, RBM42, HNRNPH2, ZFP36, NAA38, SNRPN, FXR2, SAFB2, LSM7, LSM4, ZC3H4, EIF3G, DKC1, PPIL4, RBM39, ANKHD1, KHDRBS3, RBM8A, LIN28, GRSF1, ANKRD17, UNK, NIP7, TOE1, RBM38, SRRM1, MKRN1, EIF2S1, THUMPD3, RBMX2, PUM1, MSI1, SYNCRIP, ZC3H10, HNRNPA1, RC3H1, IGF2BP3, NUPL2, SFRS1, TRA2B, CPEB2, ALKBH8, SLTM, PNPT1, THUMPD2, ASCC1, SSB, HNRNPD, LARP1B, G3BP2, ZCRB1, SNRPF, SETD1B, RBM26, MBNL2, RNF113B, NOVA1, BRUNOL6, DUS3L, SF3B4, LGTN, HNRPLL, SNRPG, ZC3H8, RBMS3, G3BP1, NONO, RBMX, A1CF, PPRC1, PDCD11, TUT1, CUGBP1, RPS3, ZC3H12C, CPSF7, YTHDF1, PABPC3, TIAL1, RBM46, UHMK1, BOLL, ZFP36L2, PAN3, MBNL1, HNRPDL, CARHSP1, RBMS1, SFRS12, MSI2, SFRS13B, C4orf23, MKI67IP, LARP1, RBM45, PPARGC1B, LSM11, SFRS15, RBPMS, ZC3H18, SYNJ1, IGF2BP1, TNRC4, U2AF1, SAFB, CPSF4, BRUNOL5, U2AF1L4, SFRS2, LARP4, RAVER1, ELAVL4, RAVER2, FUBP1, RBM15, DHX57, TDRD10, TIPARP, RBM47, SR140, TTC14, LSM6, TRA2A, HNRNPK, ENOX2, LARP6, KIAA0430, RBPMS2, SNRPD1, SAMD14, POLR2G, SF1, HNRNPH1, RSR2, PCBP1, RBMY1F, HNRNPF, HNRNPA3, RBMXL2, LSM3, NCBP2L, CSDC2, TAF15, RBM4B, RBM4, LARP7, PABPC5, LSM1, RBMXL3, MEX3C, RBM44, CSTF2T, HNRNPA0, ZC3H12D, SAMD4B, HNRNPCL1, MKRN3, RBM15B, PUF60, TRNAU1AP, SFRS2B, MEX3D, LSM10, SNRPE, TDRKH, RBM10, LENG9, EWSR1, LSMD1, DND1, MEX3B, PCBP3, THOC4, RBM12B, SNRNP35, PABPC1L2B, RALYL, DDX53, RBM33, RBM43, RBM11, ZFP36L1, YTHDF3, RNPC3, PABPC1L2A, DAZ3, RDM1, LIN28B, CPSF4L, DAZ1, ZC3H6, SFRS13A, RBM34, RRP7A, ELAVL3, C14orf21, PCBP2, ZGPAT, HNRNPAB, NOL8, HELZ, YTHDF2, SFMBT2, L3MBTL3, MEX3A, RBM20, DPPA5, MIR1236, PPP1R10, PRR3, PABPN1L, RNPS1, DAZ2, ZRSR1, RBM16, RBMXL1, CPEB1, RBMY1J, SNRPEL1, MCTS1, PABPC4L, RBMY1A1, MKRNP5, RBM14, NSUN6, UNKL, RBMY1E, HNRNPA1L2, RBMY1B, RBMY1D, RBM12, DAZ4, DGKQ, RBM17, MCM3AP, PARN, TLR2, PARP10, MOV10L1, CCAR1, LEMD3, UPF3B, RCAN2, SRRT, ZC3H12A, ZC3HAV1L, PARP14, TMEM63A, SLBP, GTF3A, NUFIP1, SRRM2, APTX, ZFR, UPF1, KIN, ZNF239, ZNF74, SNRPC, Clorf25, ZFR2, IREB2, ACO1, TROVE2, TEP1, GAPDH, and ZRANB2.

In some embodiments, the polypeptide or protein is a DNA-binding protein. Non-limiting examples of a DNA-binding protein include a chromatin-binding protein, a histone-binding protein, a histone (e.g., H1, H2A, H2B, H3, and H4), a transcription factor, a DNA replication factor, a nuclease, a chromatin modifier, and a repair factor. In some embodiments, the DNA-binding protein has a DNA-binding domain. Non-limiting examples of a DNA-binding domain include a zinc finger domain, a helix-turn-helix motif, a winged helix domain, a winged helix-turn-helix domain, a helix-loop-helix domain, a leucine zipper domain, a High Mobility Group (HMG)-box domain, a Wor3 domain, an OB-fold domain, an immunoglobulin domain, and a B3 domain.

In some embodiments, a binding agent as described herein includes an antibody. Antibodies can comprise, for example, Fab′ fragments, Fd′ fragments, Fd fragments, isolated CDRs, single chain Fvs, polypeptide-Fc fusions, single domain antibodies (e.g., shark single domain antibodies such as IgNAR or fragments thereof), camelid antibodies, single chain or Tandem diabodies (TandAb®), Anticalins®, Nanobodies® minibodies, BiTE®s, or ankyrin repeat proteins or DARPINs®. In some embodiments, the antibody is a BiTe, a (scFv)₂, a nanobody, a nanobody-HSA, a DART, a TandAb, a scDiabody, a scDiabody-CH₃, scFv-CH-CL-scFv, a HSAbody, scDiabody-HSA, or a tandem-scFv.

In some embodiments, the antibody binds or specifically binds a polypeptide of the polypeptide-nucleic acid complex or a protein of the protein-nucleic acid complex. In some embodiments, the binding agent binds the polypeptide or protein that interacts with the nucleic acid. In some embodiments, the binding agent binds a different polypeptide or protein than the polypeptide or protein that interacts with the nucleic acid (e.g., when the polypeptide-nucleic acid complex or protein-nucleic acid complex includes more than one polypeptide and/or protein).

In some embodiments, the binding agent comprises a binding moiety. Non-limiting examples of a binding moiety include biotin, polyhistidine (e.g., hexahistidine or pentahistidine), glutathione-S-transferase (GST), c-myc, calmodulin-binding peptide (CBP), FLAG, maltose-binding protein (MBP), S-tag, HA tag, green fluorescent protein (GFP), and Strep II.

Non-limiting examples of a capture moiety include avidin, streptavidin, a metal ion (e.g., Ni²⁺, Co²⁺, Cu²⁺, Zn²⁺, or Fe³⁺), glutathione, an antibody, calmodulin, a modified streptavidin, amylose, and S-protein of RNase A.

In some embodiments, the binding moiety is biotin and the capture moiety is streptavidin. In some embodiments, the binding moiety is polyhistidine and the capture moiety is a metal ion (e.g., Ni²⁺, Co²⁺, Cu²⁺, Zn²⁺, or Fe³⁺). In some embodiments, the binding moiety is GST and the capture moiety is glutathione. In some embodiments, the binding moiety is c-myc and the capture moiety is an antibody (e.g., an anti-myc antibody such as 9E10). In some embodiments, the binding moiety is CBP and the capture moiety is calmodulin. In some embodiments, the binding moiety is FLAG and the capture moiety is an antibody (e.g., an anti-FLAG antibody). In some embodiments, the binding moiety is Strep II and the capture moiety is a modified streptavidin. In some embodiments, the binding moiety is MBP and the capture moiety is amylose. In some embodiments, the binding moiety is S-tag and the capture moiety is S-protein of RNase A. In some embodiments, the binding moiety is HA tag and the capture moiety is an antibody (e.g., anti-HA antibody). In some embodiments, the binding moiety is GFP and the capture moiety is an antibody (e.g., anti-GFP antibody).

### Kits

The present disclosure also features kits useful for the spatial detection of polypeptide-nucleic acid complexes in a biological sample (e.g., a tissue section). Thus, provided herein are kits including: (a) an array including a plurality of capture probes, wherein a capture probe of the plurality of capture probes comprises: (i) a spatial barcode and (ii) a capture domain; (b) a substrate comprising a plurality of capture moieties, wherein a capture moiety of the plurality of capture moieties specifically binds a binding moiety; (c) a plurality of binding agents, wherein a binding agent specifically binds a polypeptide of a polypeptide-nucleic acid complex and comprises the binding moiety; and (d) instructions for performing any of the methods described herein.

Also provided herein are kits including: (a) an array including: (i) a plurality of capture probes, wherein a capture probe of the plurality of capture probes comprises: (i) a spatial barcode and (ii) a capture domain; (ii) a plurality of capture moieties, wherein a capture moiety of the plurality of capture moieties specifically binds a binding agent; (b) a plurality of binding agents, wherein a binding agent specifically binds a polypeptide of a polypeptide-nucleic acid complex and comprises the binding moiety; and (c) instructions for performing any of the methods described herein.

Capture probes of the spatial array can include additional domains. For example, a capture can include one or more functional domains, a cleavage domain, a unique molecular identifier (UMI), and combinations thereof. In some embodiments, the one or more functional domains includes a primer binding site or a sequencing specific site.

In some embodiments, the kit further includes one or more permeabilization reagents. In some embodiments, the one or more permeabilization reagents includes one or more proteases, a DNase, an RNase, a lipase, a detergent, and combinations thereof. In some embodiments, the one or more proteases includes one or more of pepsin, proteinase K, and collagenase.

In some embodiments, the kit further includes a polymerase. In some embodiments, the polymerase includes a reverse transcriptase and/or a DNA polymerase. Any suitable DNA polymerase can be included in the kits described herein. For example, in some embodiments, the DNA polymerase includes Bst3 polymerase and/or Klenow polymerase. Any suitable reverse transcriptase can be included in the kits described herein. Non-limiting examples of reverse transcriptases include KOD reverse transcriptase or Moloney Murine Leukemia Virus (M-MulV) reverse transcriptase.

In some embodiments, the kit further includes a hybridization buffer. In some embodiments, the kit further includes a wash buffer.

In some embodiments, the kit includes a ligase. Non-limiting examples of ligases include a PBCV-1 ligase, a Chlorella DNA ligase, a single stranded DNA ligase, and/or a T4 DNA ligase.

In some embodiments, the kit includes a reducing agent. In some embodiments, the reducing agent includes tris carboxy ethyl phosphene (TCEP) or dithiothreitol (DTT).

### Compositions

The present disclosure also features compositions in addition to the methods and kits described herein useful for the spatial detection of polypeptide-nucleic acid complexes in a biological sample (e.g., a tissue section). Thus, provided herein are compositions including: (a) an array comprising a plurality of capture probes, wherein a capture probe of the plurality of capture probes comprises: (i) a spatial barcode and (ii) a capture domain; and (b) a substrate comprising a plurality of capture moieties, wherein a capture moiety of the plurality of capture moieties is specifically bound to a binding moiety of a binding agent-polypeptide-nucleic acid complex.

Also provided herein are compositions including: (a) an array comprising: (i) a plurality of capture probes, wherein a capture probe of the plurality of capture probes comprises: (i) a spatial barcode and (ii) a capture domain; and (ii) a plurality of capture moieties, wherein a capture moiety of the plurality of capture moieties is specifically bound to a binding moiety of a binding agent-polypeptide-nucleic acid complex.

Also provided herein are compositions including: (a) an array comprising a plurality of capture probes, wherein a capture probe of the plurality of capture probes comprises: (i) a spatial barcode and (ii) a capture domain, wherein the capture domain is hybridized to a released nucleic acid of a binding agent-polypeptide-nucleic acid complex; and (b) a substrate comprising a plurality of capture moieties, wherein a capture moiety of the plurality of capture moieties is specifically bound to a binding moiety of a binding agent-polypeptide complex.

Also provided herein are compositions including: an array comprising: (i) a plurality of capture probes, wherein a capture probe of the plurality of capture probes comprises: (i) a spatial barcode and (ii) a capture domain, wherein the capture domain is hybridized to a released nucleic acid of a binding agent-polypeptide-nucleic acid complex; and (ii) a plurality of capture moieties, wherein a capture moiety of the plurality of capture moieties is specifically bound to a binding moiety of a binding agent-polypeptide complex.

Capture probes of the array can include additional domains. For example, a capture can include one or more functional domains, a cleavage domain, a unique molecular identifier (UMI), and combinations thereof. In some embodiments, the one or more functional domains includes a primer binding site or a sequencing specific site.

In some embodiments, the composition further includes one or more permeabilization reagents. In some embodiments, the one or more permeabilization reagents includes one or more proteases, a DNase, an RNase, a lipase, a detergent, and combinations thereof. In some embodiments, the one or more proteases includes one or more of pepsin, proteinase K, and collagenase.

In some embodiments, the composition further includes a polymerase. In some embodiments, the polymerase includes a reverse transcriptase and/or a DNA polymerase. Any suitable DNA polymerase can be included in the composition described herein. For example, in some embodiments, the DNA polymerase includes Bst3 polymerase and/or Klenow polymerase. Any suitable reverse transcriptase can be included in the composition described herein. Non-limiting examples of reverse transcriptases include KOD reverse transcriptase or Moloney Murine Leukemia Virus (M-MulV) reverse transcriptase.

In some embodiments, the composition includes a protease. In some embodiments, the protease includes one or more of pepsin, Proteinase K, and collagenase. In some embodiments, the composition includes a DNase.

In some embodiments, the composition includes a reducing agent. In some embodiments, the reducing agent includes tris carboxy ethyl phosphene (TCEP) or dithiothreitol (DTT).

In some embodiments, the composition includes a hybridization buffer. In some embodiments, the composition includes a wash buffer.

### EXAMPLES

### Example 1

### Methods of Spatially Detecting a Polypeptide-Nucleic Acid Complex in a Biological Sample

The present disclosure features exemplary methods of spatially detecting polypeptide-nucleic acid complexes in a biological sample (e.g., a tissue section). A biological sample (e.g., a tissue sample or a tissue section) is contacted with a binding agent (e.g., a binding agent comprising an antibody or antigen-binding fragment thereof and a binding moiety). Binding of the binding agent to a polypeptide of the polypeptide-nucleic acid complex generates a binding agent-polypeptide-nucleic acid complex.

Next, the biological sample is aligned with a first substrate comprising a plurality of capture moieties. The biological sample is aligned such that at least a portion of the biological sample is aligned with at least a portion of the substrate comprising the plurality of capture moieties (e.g., streptavidin). The binding moiety (e.g., biotin) of the binding agent in the binding agent-polypeptide-nucleic acid complex from the biological sample interacts with a capture moiety of the plurality of capture moieties. The binding moiety can be a biotin moiety and the capture moiety can be a streptavidin moiety; the biotin moiety is capable of interacting with an immobilized streptavidin molecule such that the polypeptide-nucleic acid complex is captured by the capture moiety on the first substrate through the biotin-containing binding agent.

Next, the substrate containing the captured binding agent-polypeptide-nucleic acid complex is then aligned with a spatial array comprising a plurality of capture probes where a capture probe of the plurality of capture probes comprises a spatial barcode and a capture domain. In some embodiments, a capture sequence is incorporated at an end (e.g., a 3′ end) of the nucleic acid in the complex. The nucleic acid of the binding agent-polypeptide-nucleic acid complex bound to the capture moiety is released, thereby generating a released nucleic acid. The resulting released nucleic acid is captured by a capture probe on the spatial array.

The sequence of the spatial barcode, or a complement thereof, and all or a portion of the sequence of the released nucleic acid, or a complement thereof, are determined (e.g., from an extended capture probe, or a complement or amplicon thereof, wherein the extended capture probe includes the spatial barcode and a sequence complementary to all or a portion of the released nucleic acid). These sequences are then used to determine the location of the released nucleic acid in the biological sample, thereby determining the location of the polypeptide-nucleic acid complex in the biological sample.

### Embodiments

Embodiment 1 is a method of determining a location of a polypeptide-nucleic acid complex in a biological sample, the method comprising: (a) contacting a plurality of binding agents with the biological sample, wherein a binding agent of the plurality of binding agents specifically binds a polypeptide of the polypeptide-nucleic acid complex thereby forming a binding agent-polypeptide-nucleic acid complex and wherein the binding agent comprises a binding moiety; (b) aligning the biological sample with a first substrate comprising a plurality of capture moieties such that at least a portion of the biological sample is aligned with at least a portion of the first substrate comprising the plurality of capture moieties, wherein the binding moiety of the binding agent interacts with a capture moiety of the plurality of capture moieties; (c) providing an array comprising a plurality of capture probes, wherein a capture probe of the plurality of capture probes comprises: (i) a spatial barcode, and (ii) a capture domain; (d) releasing the nucleic acid of the binding agent-polypeptide-nucleic acid complex, thereby generating a released nucleic acid; (e) hybridizing the released nucleic acid to the capture domain of the capture probe; and (f) determining (i) the sequence of the spatial barcode, or a complement thereof, and (ii) all or a portion of the sequence of the released nucleic acid, or a complement thereof, and using the determined sequences of (i) and (ii) to determine the location of the released nucleic acid in the biological sample, thereby determining the location of the polypeptide-nucleic acid complex in the biological sample.

Embodiment 2 is a method of determining a location of a protein-nucleic acid complex in a biological sample, the method comprising: (a) contacting a plurality of binding agents with the biological sample, wherein a binding agent of the plurality of binding agents specifically binds a protein of a protein-nucleic acid complex thereby forming a binding agent-protein-nucleic acid complex and wherein the binding agent comprises a binding moiety; (b) aligning the biological sample with a first substrate comprising a plurality of capture moieties such that at least a portion of the biological sample is aligned with at least a portion of the first substrate comprising the plurality of capture moieties, wherein the binding moiety of the binding agent interacts with a capture moiety of the plurality of capture moieties; (c) providing an array comprising a plurality of capture probes, wherein a capture probe of the plurality of capture probes comprises: (i) a spatial barcode, and (ii) a capture domain; (d) releasing the nucleic acid of the binding agent-protein-nucleic acid complex, thereby generating a released nucleic acid; (e) hybridizing the released nucleic acid to the capture domain of the capture probe; and (f) determining (i) the sequence of the spatial barcode, or a complement thereof, and (ii) all or a portion of the sequence of the released nucleic acid, or a complement thereof, and using the determined sequences of (i) and (ii) to identify the location of the released nucleic acid in the biological sample, thereby identifying the location of the protein-nucleic acid complex in the biological sample.

Embodiment 3 is the method of embodiment 1 or 2, wherein the biological sample is disposed on the first substrate comprising the plurality of capture moieties.

Embodiment 4 is the method of embodiment 1 or 2, wherein the biological sample is disposed on a second substrate.

Embodiment 5 is the method of embodiment 4, wherein the method further comprises aligning the second substrate comprising the biological sample with the first substrate comprising the plurality of capture moieties, such that at least a portion of the biological sample is aligned with at least a portion of the first substrate comprising the plurality of capture moieties.

Embodiment 6 is the method of any one of embodiments 1, 2, 4, or 5, wherein aligning the biological sample with the first substrate comprising the plurality of capture moieties occurs after contacting the plurality of binding agents with the biological sample.

Embodiment 7 is the method of any one of embodiments 1-6, further comprising permeabilizing the biological sample.

Embodiment 8 is the method of embodiment 7, wherein permeabilizing comprises use of one or more proteases.

Embodiment 9 is the method of embodiment 8, wherein the one or more proteases comprises pepsin, proteinase K, and collagenase.

Embodiment 10 is the method of any one of embodiments 1-9, wherein the method further comprises migrating the released nucleic acid to the array, and optionally, the migrating comprises electrophoresis.

Embodiment 11 is the method of any one of embodiments 1-10, further comprising aligning a third substrate comprising the array with the first substrate comprising the plurality of capture moieties, wherein the capture moiety is specifically bound to the binding agent of the binding agent-polypeptide-nucleic acid complex or the binding agent-protein-nucleic acid complex.

Embodiment 12 is the method of any one of embodiments 1-11, wherein when the capture moiety is specifically bound to the binding agent of the binding agent-polypeptide-nucleic acid complex or the binding agent-protein-nucleic acid complex, the method further comprises aligning the first substrate comprising the plurality of capture moieties with the a third substrate comprising the array, such that at least a portion of the first substrate comprising the plurality of capture moieties is aligned with at least a portion of the array.

Embodiment 13 is the method of any one of embodiments 1-12, wherein the first substrate comprising the plurality of capture moieties comprises fiducial markers.

Embodiment 14 is the method of any one of embodiments 11-13, wherein the third substrate comprising the array comprises fiducial markers.

Embodiment 15 is the method of any one of embodiments 1-14, wherein releasing the nucleic acid in step (d) comprises use of one or more enzymes after step (a), optionally wherein the one or more enzymes digests the binding agent and/or the polypeptide of the binding agent-polypeptide-nucleic acid complex or the binding agent and/or the protein of the binding agent-protein-nucleic acid complex.

Embodiment 16 is the method of any one of embodiments 1-14, wherein releasing the nucleic acid in step (d) comprises use of one or more enzymes after step (b), optionally wherein the one or more enzymes digests the binding agent and/or the polypeptide of the binding agent-polypeptide-nucleic acid complex or the binding agent and/or the protein of the binding agent-protein-nucleic acid complex.

Embodiment 17 is the method of embodiment 15 or 16, wherein the one or more enzymes comprise pepsin or proteinase K.

Embodiment 18 is the method of any one of embodiments 1-17, wherein the capture domain of the capture probe comprises a poly(T) sequence.

Embodiment 19 is the method of any one of embodiments 1-17, wherein the capture domain of the capture probe comprises a fixed sequence.

Embodiment 20 is the method of any one of embodiments 1-19, wherein the capture probe is extended using the released nucleic acid as a template, thereby generating an extended capture probe.

Embodiment 21 is the method of any one of embodiments 1-20, wherein the released nucleic acid is extended using the capture probe as a template.

Embodiment 22 is the method of any one of embodiments 1-21, wherein the determining step comprises sequencing.

Embodiment 23 is the method of embodiment 22, wherein the sequencing comprises high-throughput sequencing.

Embodiment 24 is the method of any one of embodiments 1-23, wherein the biological sample is fixed.

Embodiment 25 is the method of embodiment 24, wherein the biological sample is methanol-fixed, acetone-fixed, paraformaldehyde-fixed, or is formalin-fixed and paraffin-embedded (FFPE).

Embodiment 26 is the method of any one of embodiments 1-25, wherein the method further comprises staining the biological sample.

Embodiment 27 is the method of embodiment 26, wherein the staining comprises use of immunofluorescence, immunohistochemistry, or hematoxylin and/or eosin.

Embodiment 28 is the method of any one of embodiments 1-27, wherein the method further comprises imaging the biological sample.

Embodiment 29 is the method of any one of embodiments 1-28, wherein the biological sample is a tissue sample.

Embodiment 30 is the method of embodiment 29, wherein the tissue sample is a fixed tissue sample.

Embodiment 31 is the method of embodiment 30, wherein the fixed tissue sample is a methanol-fixed tissue sample, an acetone-fixed tissue sample, a paraformaldehyde tissue sample, or a formalin-fixed paraffin-embedded tissue sample.

Embodiment 32 is the method of embodiment 29, wherein the tissue sample is a fresh-frozen tissue sample.

Embodiment 33 is the method of any one of embodiments 1-28, wherein the biological sample is a tissue section.

Embodiment 34 is the method of embodiment 33, wherein the tissue section is a fixed tissue section.

Embodiment 35 is the method of embodiment 34, wherein the fixed tissue section is a methanol-fixed tissue section, an acetone-fixed tissue section, a paraformaldehyde tissue section, or a formalin-fixed paraffin-embedded tissue section.

Embodiment 36 is the method of embodiment 33, wherein the tissue section is a fresh-frozen tissue section.

Embodiment 37 is the method of any one of embodiments 1 or 3-36, wherein the polypeptide-nucleic acid complex comprises two or more polypeptides.

Embodiment 38 is the method of any one of embodiments 1 or 3-36, wherein the polypeptide-nucleic acid complex comprises one polypeptide.

Embodiment 39 is the method of any one of embodiments 2-36, wherein the protein-nucleic acid complex comprises two or more proteins.

Embodiment 40 is the method of any one of embodiments 2-36, wherein the protein-nucleic acid complex comprises one protein.

Embodiment 41 is the method of any one of embodiments 1-40, wherein the polypeptide or protein comprises one or more of a ribosomal protein, a ribozyme, a histone, a transcription factor, a DNA-binding protein, and an RNA-binding protein.

Embodiment 42 is the method any one of embodiments 1-41, wherein the polypeptide-nucleic acid complex or the protein-nucleic acid complex comprises a ribonucleoprotein.

Embodiment 43 is the method of any one of embodiments 1-42, wherein the released nucleic acid comprises RNA.

Embodiment 44 is the method of embodiment 43, wherein the RNA is an mRNA.

Embodiment 45 is the method of any one of embodiments 1-42, wherein the released nucleic acid comprises DNA.

Embodiment 46 is the method of embodiment 45, wherein the DNA is genomic DNA.

Embodiment 47 is the method of any one of embodiments 1-45, wherein the method further comprises incorporating a capture sequence onto an end of the nucleic acid of the binding agent-polypeptide-nucleic acid complex or the nucleic acid of the binding agent-protein-nucleic acid complex.

Embodiment 48 is the method of embodiment 47, wherein incorporating the capture sequence onto the end of the nucleic acid of the binding agent-polypeptide-nucleic acid complex or the nucleic acid of the binding agent-protein-nucleic acid complex comprises incorporating a poly(A) oligonucleotide onto the end of the nucleic acid of the binding agent-polypeptide-nucleic acid complex or the nucleic acid of the binding agent-protein-nucleic acid complex, optionally wherein the incorporating comprises ligating.

Embodiment 49 is the method of embodiment 47, wherein incorporating the capture sequence onto the end of the nucleic acid of the binding agent-polypeptide-nucleic acid complex or the nucleic acid of the binding agent-protein-nucleic acid complex comprises the use of a ligase, a poly(A) polymerase, a terminal transferase, a plurality of dATPs, or a combination thereof.

Embodiment 50 is the method of any one of embodiments 1-49, wherein the binding moiety comprises biotin.

Embodiment 51 is the method of any one of embodiments 1-50, wherein the capture moiety comprises avidin or streptavidin.

Embodiment 52 is the method of any one of embodiments 1-51, wherein the capture probe further comprises one or more functional domains, a cleavage domain, a unique molecular identifier, and combinations thereof.

Embodiment 53 is the method of embodiment 52, wherein the one or more functional domains comprises a primer binding site or a sequencing specific site.

Embodiment 54 is a method of determining a location of a polypeptide-nucleic acid complex in a biological sample, the method comprising: (a) aligning the biological sample with a substrate comprising: (i) a plurality of capture probes, wherein a capture probe of the plurality of capture probes comprises: (i) a spatial barcode and (ii) a capture domain; and (ii) a plurality of capture moieties, wherein a capture moiety specifically binds a binding moiety; (b) contacting a plurality of binding agents with the biological sample, wherein a binding agent of the plurality of binding agents specifically binds a polypeptide of the polypeptide-nucleic acid complex thereby forming a binding agent-polypeptide-nucleic acid complex; (c) binding the binding agent of the binding agent-polypeptide-nucleic acid complex to the capture moiety of the plurality of capture moieties; (d) releasing the nucleic acid of the binding agent-polypeptide-nucleic acid complex, thereby generating a released nucleic acid; (e) hybridizing the released nucleic acid to the capture domain of the capture probe; and (f) determining (i) the sequence of the spatial barcode, or a complement thereof, and (ii) all or a portion of the sequence of the released nucleic acid, or a complement thereof, and using the determined sequences of (i) and (ii) to determine the location of the released nucleic acid in the biological sample, thereby determining the location of the polypeptide-nucleic acid complex in the biological sample.

Embodiment 55 is a kit comprising: (a) an array comprising a plurality of capture probes, wherein a capture probe of the plurality of capture probes comprises: (i) a spatial barcode and (ii) a capture domain; (b) a substrate comprising a plurality of capture moieties, wherein a capture moiety of the plurality of capture moieties specifically binds a binding moiety; (c) a plurality of binding agents, wherein a binding agent specifically binds a polypeptide of a polypeptide-nucleic acid complex and comprises the binding moiety; and (d) instructions for performing any of the methods of embodiments 1-47.

Embodiment 56 is a kit comprising: (a) an array comprising: (i) a plurality of capture probes, wherein a capture probe of the plurality of capture probes comprises: (i) a spatial barcode and (ii) a capture domain; (ii) a plurality of capture moieties, wherein a capture moiety of the plurality of capture moieties specifically binds a binding agent; (b) a plurality of binding agents, wherein a binding agent specifically binds a polypeptide of a polypeptide-nucleic acid complex and comprises the binding moiety; and (c) instructions for performing the method of embodiment 48.

Embodiment 57 is the kit of embodiment 55 or 56, wherein the kit further comprises one or more permeabilization reagents.

Embodiment 58 is the kit of embodiment 57, wherein the one or more permeabilization reagents comprises one or more proteases, a DNase, an RNase, a lipase, a detergent, and combinations thereof.

Embodiment 59 is the kit of embodiment 58, wherein the one or more proteases comprise pepsin, proteinase K, and collagenase.

Embodiment 60 is the kit of any one of embodiments 55-59, wherein the capture probe further comprises one or more functional domains, a cleavage domain, a unique molecular identifier, and combinations thereof.

Embodiment 61 is the kit of embodiment 60, wherein the one or more functional domains comprises a primer binding site or a sequencing specific site.

Embodiment 62 is the kit of any one of embodiments 55-61, further comprising a polymerase.

Embodiment 63 is the kit of embodiment 62, wherein the polymerase comprises a reverse transcriptase and/or a DNA polymerase.

Embodiment 64 is a composition comprising: (a) an array comprising a plurality of capture probes, wherein a capture probe of the plurality of capture probes comprises: (i) a spatial barcode and (ii) a capture domain; and (b) a substrate comprising a plurality of capture moieties, wherein a capture moiety of the plurality of capture moieties is specifically bound to a binding moiety of a binding agent-polypeptide-nucleic acid complex.

Embodiment 65 is a composition comprising: (a) an array comprising: (i) a plurality of capture probes, wherein a capture probe of the plurality of capture probes comprises: (i) a spatial barcode and (ii) a capture domain; and (ii) a plurality of capture moieties, wherein a capture moiety of the plurality of capture moieties is specifically bound to a binding moiety of a binding agent-polypeptide-nucleic acid complex.

Embodiment 66 is a composition comprising: (a) an array comprising a plurality of capture probes, wherein a capture probe of the plurality of capture probes comprises: (i) a spatial barcode and (ii) a capture domain, wherein the capture domain is hybridized to a released nucleic acid of a binding agent-polypeptide-nucleic acid complex; and (b) a substrate comprising a plurality of capture moieties, wherein a capture moiety of the plurality of capture moieties is specifically bound to a binding moiety of a binding agent-polypeptide complex.

Embodiment 67 is a composition comprising: an array comprising: (i) a plurality of capture probes, wherein a capture probe of the plurality of capture probes comprises: (i) a spatial barcode and (ii) a capture domain, wherein the capture domain is hybridized to a released nucleic acid of a binding agent-polypeptide-nucleic acid complex; and (ii) a plurality of capture moieties, wherein a capture moiety of the plurality of capture moieties is specifically bound to a binding moiety of a binding agent-polypeptide complex.

Embodiment 68 is the composition of any one of embodiments 64-67, wherein the composition further comprises one or more permeabilization reagents.

Embodiment 69 is the composition of embodiment 68, wherein the one or more permeabilization reagents comprises one or more proteases, a DNase, an RNase, a lipase, a detergent, and combinations thereof.

Embodiment 70 is the composition of embodiment 69, wherein the one or more proteases comprise pepsin, proteinase K, and collagenase.

Embodiment 71 is the composition of any one of embodiments 64-70, wherein the capture probe further comprises one or more functional domains, a cleavage domain, a unique molecular identifier (UMI), and combinations thereof.

Embodiment 72 is the composition of embodiment 71, wherein the one or more functional domains comprises a primer binding site or a sequencing specific site.

Embodiment 73 is the composition of any one of embodiments 64-72, further comprising a polymerase.

Embodiment 74 is the composition of embodiment 73, wherein the polymerase comprises a reverse transcriptase and/or a DNA polymerase.

What is claimed is:

1. A method of determining a location of a polypeptide-nucleic acid complex in a biological sample, the method comprising:
(a) contacting a plurality of binding agents with the biological sample, wherein a binding agent of the plurality of binding agents binds a polypeptide of the polypeptide-nucleic acid complex in the biological sample thereby forming a binding agent-polypeptide-nucleic acid complex and wherein the binding agent comprises a binding moiety;
(b) aligning the biological sample with a first substrate comprising a plurality of capture moieties such that at least a portion of the biological sample is aligned with at least a portion of the first substrate comprising the plurality of capture moieties;
(c) contacting the binding moiety of the binding agent of the binding agent-polypeptide-nucleic acid complex with a capture moiety of the plurality of capture moieties, such that the binding agent-polypeptide-nucleic acid complex is captured on the first substrate;
(d) releasing the nucleic acid of the binding agent-polypeptide-nucleic acid complex captured on the first substrate, thereby generating a released nucleic acid;
(e) hybridizing the released nucleic acid to a capture domain of a capture probe comprised in an array comprising a plurality of capture probes, wherein the capture probe of the plurality of capture probes comprises: (i) a spatial barcode, and (ii) the capture domain; and
(f) determining (i) the sequence of the spatial barcode, or a complement thereof, and (ii) all or a portion of the sequence of the released nucleic acid, or a complement thereof, and using the determined sequences of (i) and (ii) to determine the location of the released nucleic acid in the biological sample, thereby determining the location of the polypeptide-nucleic acid complex in the biological sample.

2. The method of claim 1, wherein the biological sample is disposed on the first substrate comprising the plurality of capture moieties, optionally wherein aligning the biological sample with the first substrate comprising the plurality of capture moieties occurs after contacting the plurality of binding agents with the biological sample.

3. The method of claim 1, wherein the biological sample is disposed on a second substrate and the method further comprises aligning the second substrate comprising the biological sample with the first substrate comprising the plurality of capture moieties, such that at least a portion of the biological sample is aligned with at least a portion of the first substrate comprising the plurality of capture moieties.

4. The method of claim 1, further comprising permeabilizing the biological sample, optionally wherein permeabilizing comprises use of one or more proteases and/or detergents.

5. The method of claim 1, further comprising aligning a third substrate comprising the array with the first substrate comprising the binding agent-polypeptide-nucleic acid complex captured thereon, wherein the capture moiety is bound to the binding agent of the binding agent-polypeptide-nucleic acid complex, optionally wherein aligning the third substrate with the first is performed prior to releasing the nucleic acid in step (d).

6. The method of claim 5, wherein the first substrate comprising the plurality of capture moieties and/or the third substrate comprising the array comprises fiducial markers.

7. The method of claim 1, wherein releasing the nucleic acid in step (d) comprises use of one or more enzymes after (a) or (b), optionally wherein the one or more enzymes digests the binding agent and/or the polypeptide of the binding agent-polypeptide-nucleic acid complex.

8. The method of claim 7, wherein the one or more enzymes comprise pepsin or proteinase K.

9. The method of claim 1, wherein the capture domain of the capture probe comprises a fixed sequence, optionally a poly(T) sequence or and the capture probe further comprises one or more functional domains, a cleavage domain, a unique molecular identifier, or a combination thereof.

10. The method of claim 1, wherein the capture probe is extended using the released nucleic acid as a template, thereby generating an extended capture probe, and optionally wherein the released nucleic acid is extended using the capture probe as a template.

11. The method of claim 10, wherein the determining step comprises sequencing the extended capture probe, or a complement or amplicon thereof.

12. The method of claim 1, wherein the method further comprises imaging and/or staining the biological sample.

13. The method of claim 1, wherein the biological sample is a tissue sample, optionally a tissue section.

14. The method of claim 13, wherein the tissue section is a fresh-frozen tissue section or a fixed tissue section, optionally wherein the fixed tissue section is a methanol-fixed tissue section, an acetone-fixed tissue section, a paraformaldehyde tissue section, or a formalin-fixed paraffin-embedded tissue section.

15. The method claim 1, wherein the polypeptide-nucleic acid complex comprises two or more polypeptides.

16. The method claim 1, wherein the polypeptide of the polypeptide-nucleic acid complex is comprised in a protein.

17. The method of claim 16, wherein the protein comprises one or more of a ribosomal protein, a ribozyme, a histone, a transcription factor, a DNA-binding protein, and an RNA-binding protein.

18. The method of claim 1, wherein the released nucleic acid comprises DNA or RNA, optionally, wherein the RNA is mRNA.

19. The method of claim 1, wherein the method further comprises incorporating a capture sequence onto an end of the nucleic acid of the binding agent-polypeptide-nucleic acid complex, wherein incorporating the capture sequence comprises incorporating a poly(A) oligonucleotide onto the end of the nucleic acid of the binding agent-polypeptide-nucleic acid complex.

20. The method of claim 19, wherein incorporating the capture sequence onto the end of the nucleic acid of the binding agent-polypeptide-nucleic acid complex comprises the use of a ligase, a poly(A) polymerase, a terminal transferase, a plurality of dATPs, or a combination thereof.

21. The method of claim 1, wherein the binding moiety comprises biotin and the capture moiety comprises avidin or streptavidin.

22. The method of claim 1, further comprising migrating the released nucleic acid to the array, optionally wherein the migrating comprises electrophoresis.

23. The method of claim 1, wherein the binding agent comprises and antibody or an antigen binding fragment thereof.
